(19) United States
(12) Patent Application Publication
(10) Pub. No.: US 2011/0263670 A1
(43) Pub. Date: Oct. 27, 2011

(54) NOVEL POLYMORPHS OF SUNITINIB AND PROCESSES FOR THEIR PREPARATION

(76) Inventors: Vinayak Gore, Maharashtra (IN); Bharati Choudhari, Maharashtra (IN); Mahesh Hublikar, Maharashtra (IN); Prakash Bansode, Maharashtra (IN)

(21) Appl. No.: 13/060,322

(22) PCT Filed: Aug. 24, 2009
(86) PCT No.: PCT/GB2009/051055

§ 371 Date: Jul. 12, 2011

(30) Foreign Application Priority Data
Aug. 25, 2008 (IN) 1435/KOL/2008

(51) Int. Cl. A61K 31/404; A61P 35/00; C07D 403/06

(52) U.S. Cl. 514/414; 548/486

(57) ABSTRACT

The present invention relates to novel polymorphs of sunitinib free base designated form II and form III and to processes for their preparation. The invention also relates to their use as APIs and in the preparation of various forms of sunitinib. Further, the invention relates to pharmaceutical compositions comprising said novel polymorphs and salts, solvates and hydrates prepared according to the invention, and to the uses of said pharmaceutical compositions in the treatment and/or prevention of cancer.

### FIELD OF THE INVENTION

The present invention relates to novel polymorphs of sunitinib free base designated form II and form III and to processes for their preparation. The invention also relates to their use as APIs and in the preparation of various forms of sunitinib. Further, the invention relates to pharmaceutical compositions comprising said novel polymorphs and salts, solvates and hydrates prepared according to the invention, and to the uses of said pharmaceutical compositions in the treatment and/or prevention of cancer.

### BACKGROUND OF THE INVENTION

Sunitinib, represented by formula (I) and chemically named N[2-(diethylamino)ethyl]-5-[(Z)-(5-fluoro-2-oxo-1,2-dihydro-3H-indol-3-ylidene)methyl]-2,4-dimethyl-1H-pyrrole-3-carboxamide, is an oral tyrosine kinase inhibitor (TKI) that targets and blocks the signaling pathways of multiple selected receptor tyrosine kinases (RTKs).

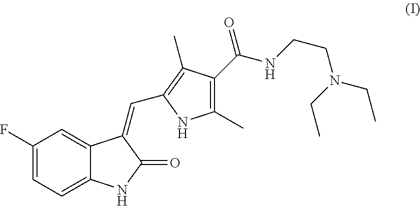

Through competitive inhibition of ATP binding sites, sunitinib inhibits the TK activity of a group of closely related RTKs, all of which are involved in various human malignancies: the vascular endothelial growth factor receptors (VEGFR-1, -2, -3), the platelet derived growth factor receptors (PDGF-R), the stem cell factor (KIT), CSF-1R, Flt3, and RET. Sunitinib is therefore useful for the treatment of cancer and tumors. It is currently marketed for the treatment of unresectable and/or metastatic malignant gastrointestinal stromal tumor (GIST) and advanced and/or metastatic renal cell carcinoma (MRCC). The product is marketed as sunitinib malate under the proprietary name Sutent®.

Sunitinib was first described in WO 2001/060814 and EP1255752 as one of a number of PK modulating compounds. The possibility of a number of salts is also disclosed therein. Such salts may include the hydrochloride, sulfate, carbonate, lactate, tartrate, malate, maleate and succinate salts. However, the disclosure is silent as to the nature of specific crystal forms of sunitinib.

WO 2003/016305 further states that although the free base may be crystallized as small particles, it is desirable in large scale operations, for example, to have larger particle size crystals for ease in filtration.

It has long been an aim of the formulation scientist to develop alternative forms of active pharmaceutical ingredients (API). These forms, which include salts, solvates and hydrates, may be used as simple alternatives to the active ingredient for cost reasons or as a means of circumventing legal issues or they may possess advantageous properties such as an improved rate of dissolution, easier manufacture, increased bioavailability, decreased toxicity or increased efficacy. For the same reasons the formulation scientist would also seek to develop new polymorphs of an API.

Polymorphs are distinct solids sharing the same molecular formula, yet each polymorph may have distinct physical properties. Therefore a single compound may give rise to a variety of polymorphic forms where each form has different and distinct physical properties, such as different solubility profiles, different melting point temperatures and/or different X-ray diffraction peaks. The solubility of each polymorph may vary and consequently identifying the existence of polymorphs of an API is essential for providing pharmaceutical compositions with predictable solubility profiles. Polymorphic forms of a compound can be distinguished in a laboratory by X-ray diffraction spectroscopy and by other methods such as infrared spectrometry. Additionally, the properties of polymorphic forms of the same active pharmaceutical ingredient are well known in the pharmaceutical art to have an effect on the manufacture of drug product compositions comprising the API. For example, the solubility, stability, flowability, tractability and compressibility of the API as well as the safety and efficacy of the drug product can be dependent on the polymorphic form.

The discovery of new polymorphic forms of a pharmaceutically useful compound provides an opportunity to improve the performance characteristics of a pharmaceutical product. It also adds to the material that a formulation scientist has available for designing, for example, a pharmaceutical dosage form of a drug with a targeted release profile or other desired characteristic. When a polymorphic form has been discovered to be useful and advantageous, either in the preparation of a pharmaceutical composition or as an intermediate in the preparation of another active pharmaceutical ingredient (API), the next challenge is to develop methods of synthesis that are simple and cost effective and provide the desired polymorph in the purest form possible.

### SUMMARY OF THE INVENTION

Therefore, it is an object of the present invention to provide novel polymorphs of sunitinib with improved properties that are suitable for large scale production. Improved properties may include improved solubility, bioavailability, stability including chemical and polymorphic stability, flowability, tractability, compressibility, compactability, toxicity, efficacy or safety.

It is a further object of the present invention to provide processes to produce the novel polymorphs.

It is a still further object of the present invention to provide pharmaceutical compositions containing the novel polymorphs.

The inventors have developed novel polymorphs of sunitinib free base that are anhydrous, crystalline, non-hygroscopic, stable and suitable for large scale preparation.

Accordingly, in a first aspect of the present invention there is provided a crystalline form II of sunitinib having a characteristic XRPD spectrum comprising two or more peaks (preferably three or more, four or more, five or mote, six or more, seven or more, eight or more, nine or more, ten or more, eleven or more, twelve or more, thirteen or more, fourteen or more, or fifteen peaks) with 2θ values at 3.76, 4.38, 7.65, 8.93, 10.17, 11.57, 16.46, 17.92, 20.15, 25.69, 26.39, 27.83, 28.40, 40.04 and 49.65±0.2 °2θ, when Cu α-radiation is used. Preferably the present invention provides a crystalline form II of sunitinib having a characteristic XRPD spectrum comprising two or more major peaks with 2θ values at 3.76, 4.38, 7.65, 8.93, 10.17, 11.57, 16.46, 17.92, 20.15, 25.69, 26.39, 27.83, 28.40, 40.04 and 49.65±0.2 °2θ, when Cu α-radiation is used. A particularly preferred embodiment comprises crystalline form II of sunitinib having an XRPD spectrum substantially as shown in FIG. 1.

Preferably the crystalline form II according to the first aspect of the invention is further characterized by a differential scanning calorimetry (DSC) with an endothermic peak at about 238° C., preferably with endothermic peaks at about 212° C., 225° C. and 238° C., when a rate of heating of 10° C./min is used. Preferably the sunitinib form II has a DSC trace substantially as shown in FIG. 2.

In another embodiment, the crystalline form II according to the first aspect of the invention is further characterized by a thermogravimetric analysis (TGA) loss of 0% over a range of between about 25-220° C., when a rate of heating of 10° C./min is used. Preferably the sunitinib form II has a TGA trace substantially as shown in FIG. 3.

Preferably the crystalline form II of sunitinib according to the first aspect of the invention is anhydrous. In one embodiment the anhydrous form II comprises less than about 5%, more preferably less than about 4%, and most preferably less than about 2% water. In further embodiments the form II is non-hygroscopic and stable.

Preferably the crystalline form II of sunitinib has a chemical purity of greater than 99%, preferably greater than 99.3%, more preferably greater than 99.4%, even more preferably greater than 99.5%, yet more preferably greater than 99.6%, yet more preferably still greater than 99.7%, most preferably greater than 99.9%, preferably as measured by HPLC.

Preferably the crystalline form II of sunitinib has a polymorphic purity of greater than 98%, preferably greater than 99%, preferably greater than 99.3%, more preferably greater than 99.4%, even more preferably greater than 99.5%, yet more preferably greater than 99.6%, yet more preferably still greater than 99.7%, most preferably greater than 99.9%, preferably as measured by XRPD or DSC, preferably as measured by XRPD.

According to a second aspect of the present invention there is provided a process for the preparation of crystalline form II of sunitinib, comprising the steps of:

(a) dissolving or suspending sunitinib in a solvent;
(b) causing crystalline form II of sunitinib to precipitate from the solution or suspension obtained in step (a); and
(c) isolating the solid obtained in step (b).

Preferably sunitinib is dissolved in step (a).

Preferably the solvent in step (a) is a non-hydroxylic solvent. Preferably the non-hydroxylic solvent in step (a) is an ester. Preferred esters are esters R—COOR, wherein each R is independently C₁-C₆ alkyl, C₆-C₁₀ arylalkyl or C₆-C₁₀ aryl, each of which may optionally be substituted. Preferably the ester is methyl acetate, ethyl acetate, n-propyl acetate, isopropyl acetate, n-butyl acetate, sec-butyl acetate, isobutyl acetate, tert-butyl acetate, isoamyl acetate, methyl phenylacetate, ethyl butyrate, benzyl benzoate, ethyl acetoacetate, ethyl lactate, ethylene carbonate, propylene carbonate, or a mixture thereof. Most preferably, the ester is ethyl acetate.

In another embodiment, the solvent in step (a) is heated to dissolve sunitinib, most preferably the sunitinib is heated to reflux temperature. More preferably, the temperature is between about 64-68° C.

In particularly preferred embodiments, the solution from step (a) is further filtered. Most preferably, the solution obtained in step (a) is filtered under vacuum or partial vacuum.

In another particularly preferred embodiment, step (b) comprises causing form II to precipitate from the solution obtained in step (a) by cooling the solution, most preferably the solution is cooled to between about 0-5° C. Alternatively, in those embodiments where the solvent comprising sunitinib from step (a) has been heated to effect dissolution, the solution is cooled to ambient temperature, most preferably to between about 20-35° C.

In some embodiments, the solvent is allowed to evaporate to isolate the solid obtained in step (b). In alternative preferred embodiments, the reaction mixture of step (b) is filtered, preferably under vacuum. Preferably the isolated sunitinib is washed with the solvent employed in step (a). Preferably the isolated sunitinib is allowed to dry until a constant weight is achieved, preferably at about 40° C., preferably under conditions of reduced pressure, most preferably under vacuum or partial vacuum.

Preferably the process of the second aspect of the present invention is carried out on an industrial scale, preferably to obtain sunitinib form II in batches of 0.1 kg or more, 0.5 kg or more, 1 kg or more, 5 kg or more, 10 kg or more, or 50 kg or more.

Preferably the sunitinib form II is obtained in a yield of 50% or more, 60% or more, 70% or more, or 80% or more.

Preferably the sunitinib form II obtained has a chemical purity of 99% or more, 99.3% or more, 99.4% or more, 99.5% or more, 99.6% or more, 99.7% or more, or 99.9% or more, preferably as measured by HPLC.

Preferably the sunitinib form II obtained has a polymorphic purity of 98% or more, 99% or more, 99.3% or more, 99.4% or more, 99.5% or more, 99.6% or more, 99.7% or more, or 99.9% or more, preferably as measured by XRPD or DSC, preferably as measured by XRPD.

According to a third aspect of the present invention there is provided a crystalline form III of sunitinib having a characteristic XRPD spectrum comprising two or more peaks (preferably three or more, four or more, five or more, six or more, seven or more, eight or more, nine or more, ten or more, eleven or more, twelve or more, thirteen or more, or fourteen peaks) with 2θ values at 4.40, 8.91, 10.45, 14.79, 16.40, 18.00, 18.59, 20.11, 22.76, 25.56, 27.81, 32.31, 40.02 and 49.71±0.2 °2θ, when Cu α-radiation is used. Preferably the present invention provides a crystalline form III of sunitinib having a characteristic XRPD spectrum comprising two or more major peaks with 2θ values at 4.40, 8.91, 10.45, 14.79, 16.40, 18.00, 18.59, 20.11, 22.76, 25.56, 27.81, 32.31, 40.02 and 49.71±0.2 °2θ, when Cu α-radiation is used. A particularly preferred embodiment comprises crystalline form III of sunitinib have an XRPD spectrum substantially as shown in FIG. 4.

Preferably the crystalline form III according to the third aspect of the invention is further characterized by a differential scanning calorimetry (DSC) with an endothermic peak at about 239° C., when a rate of heating of 10° C./min is used. Preferably the sunitinib form III has a DSC trace substantially as shown in FIG. 5.

In another embodiment, the crystalline form III according to the third aspect of the invention is further characterized by a thermogravimetric analysis (TGA) loss of 0% over a range of between about 25-220° C., when a rate of heating of 10° C./min is used. Preferably the sunitinib form III has a TGA trace substantially as shown in FIG. 6.

Preferably the crystalline form III of sunitinib according to the third aspect of the invention is anhydrous. In one embodiment the anhydrous form III comprises less than about 5%, more preferably less than about 4%, and most preferably less than about 2% water. In further embodiments the form III is non-hygroscopic and stable.

Preferably the crystalline form III of sunitinib has a chemical purity of greater than 99%, preferably greater than 99.3%, more preferably greater than 99.4%, even more preferably greater than 99.5%, yet more preferably greater than 99.6%, yet more preferably still greater than 99.7%, most preferably greater than 99.9%, preferably as measured by HPLC.

Preferably the crystalline form III of sunitinib has a polymorphic purity of greater than 98%, preferably greater than 99%, preferably greater than 99.3%, more preferably greater than 99.4%, even more preferably greater than 99.5%, yet more preferably greater than 99.6%, yet more preferably still greater than 99.7%, most preferably greater than 99.9%, preferably as measured by XRPD or DSC, preferably as measured by XRPD.

According to a fourth aspect of the present invention there is provided a process for the preparation of crystalline form III of sunitinib, comprising the steps of:

(a) dissolving or suspending sunitinib in a solvent;
(b) causing crystalline form III of sunitinib to precipitate from the solution or suspension obtained in step (a); and
(c) isolating the solid obtained in step (b).

Preferably sunitinib is dissolved in step (a).

Preferably the solvent in step (a) is a non-hydroxylic solvent. Preferably the solvent is a ketone. Preferred ketones are ketones R—COR, wherein each R is independently C₁-C₆ alkyl, C₆-C₁₀ arylalkyl or C₆-C₁₀ aryl, each of which may optionally be substituted. Preferably the ketone is acetone, methyl ethyl ketone, methyl n-propyl ketone, methyl isopropyl ketone, methyl n-butyl ketone, methyl isobutyl ketone, diethyl ketone, ethyl isopropyl ketone, acetophenone, isophorone, mesityl oxide, or a mixture thereof. Most preferably, the solvent in step (a) is acetone.

In an alternative embodiment, the solvent in step (a) is an alcohol, more preferably a C₁-C₆ alcohol. Preferred alcohols are alcohols R—OH, wherein R is C₁-C₆ alkyl, C₆-C₁₀ arylalkyl or C₆-C₁₀ aryl, each of which may optionally be substituted. Preferably R is unsubstituted C₁-C₆ alkyl. Preferably the alcohol is methanol, ethanol, n-propanol, isopropanol or isopropyl alcohol (IPA), n-butanol, sec-butanol, isobutanol, tert-butanol, or a mixture thereof. In one embodiment, the alcohol is not ethanol. Most preferably, the solvent is isopropyl alcohol (IPA).

Preferably the solvent in step (a) is heated to dissolve sunitinib, most preferably the sunitinib is heated to elevated temperatures, preferably to reflux temperature, preferably the temperature is between about 54-58° C. when the solvent is acetone and between about 78-82° C. when the solvent is IPA.

In a particularly preferred embodiment, the solution obtained in step (a) is further filtered, most preferably under reduced pressure or a vacuum.

In another particularly preferred embodiment, step (b) comprises causing form III to precipitate from the solution obtained in step (a) by cooling the solution, most preferably the solution is cooled to between about 0-5° C. Alternatively, in those embodiments where the solvent comprising sunitinib from step (a) has been heated to effect dissolution, the solution is cooled to ambient temperature, most preferably between about 20-35° C.

In some embodiments, the solvent is allowed to evaporate to isolate the solid obtained in step (b). In alternative preferred embodiments, the reaction mixture of step (b) is filtered, preferably under vacuum. Preferably the isolated sunitinib is washed with the solvent employed in step (a). Preferably the isolated sunitinib is allowed to dry until a constant weight is achieved, preferably at about 40° C., preferably under conditions of reduced pressure, most preferably under vacuum or partial vacuum.

Preferably the process of the fourth aspect of the present invention is carried out on an industrial scale, preferably to obtain sunitinib form III in batches of 0.1 kg or more, 0.5 kg or more, 1 kg or more, 5 kg or more, 10 kg or more, or 50 kg or more.

Preferably the sunitinib form III is obtained in a yield of 50% or more, 60% or more, 70% or more, 80% or more, or 90% or more.

Preferably the sunitinib form III obtained has a chemical purity of 99% or more, 99.3% or more, 99.4% or more, 99.5% or more, 99.6% or more, 99.7% or more, or 99.9% or more, preferably as measured by HPLC.

Preferably the sunitinib form III obtained has a polymorphic purity of 98% or more, 99% or more, 99.3% or more, 99.4% or more, 99.5% or more, 99.6% or more, 99.7% or more, or 99.9% or more, preferably as measured by XRPD or DSC, preferably as measured by XRPD.

A fifth aspect of the present invention provides a process for preparing sunitinib malate, comprising reacting the sunitinib form II according to the first aspect of the invention or prepared by a process according to the second aspect of the invention, or the sunitinib form III according to the third aspect of the invention or prepared by a process according to the fourth aspect of the invention, with malic acid. Preferably the malic acid is L-malic acid, or alternatively the malic acid is D-malic acid.

Preferably the sunitinib form II according to the first aspect of the invention or prepared by a process according to the second aspect of the invention, or the sunitinib form III according to the third aspect of the invention or prepared by a process according to the fourth aspect of the invention, or the sunitinib malate prepared by a process according to the fifth aspect of the invention, is suitable for use in medicine, preferably for treating or preventing cancer or a tumor, more preferably for treating or preventing unresectable and/or metastatic malignant gastrointestinal stromal tumor (GIST) or advanced and/or metastatic renal cell carcinoma (MRCC).

According to a sixth aspect of the present invention there is provided a pharmaceutical composition comprising sunitinib form II or form III or sunitinib according to any of the aspects of the present invention. Preferably the pharmaceutical composition according to the sixth aspect of the invention is for use in the treatment or prevention of cancer or a tumor, more preferably the treatment or prevention of unresectable and/or metastatic malignant gastrointestinal stromal tumor (GIST) or advanced and/or metastatic renal cell carcinoma (MRCC).

According to a seventh aspect of the present invention there is provided the use of the sunitinib form II according to the first aspect of the invention or prepared by a process according to the second aspect of the invention, or the use of the sunitinib form III according to the third aspect of the invention or prepared by a process according to the fourth aspect of the invention, or the use of the sunitinib malate prepared by a process according to the fifth aspect of the invention, for the manufacture of a medicament for treating or preventing cancer or a tumor, preferably for the manufacture of a medicament for treating or preventing unresectable and/or metastatic malignant gastrointestinal stromal tumor (GIST) or advanced and/or metastatic renal cell carcinoma (MRCC).

According to an eight aspect of the present invention there is provided a method of treating or preventing cancer or a tumor, the method comprising administering to a patient in need thereof a therapeutically or prophylactically effective amount of the sunitinib form II according to the first aspect of the invention or prepared by a process according to the second aspect of the invention, or a therapeutically or prophylactically effective amount of the sunitinib form III according to the third aspect of the invention or prepared by a process according to the fourth aspect of the invention, or a therapeutically or prophylactically effective amount of the sunitinib malate prepared by a process according to the fifth aspect of the invention, or a therapeutically or prophylactically effective amount of a pharmaceutical composition according to the sixth aspect of the invention. Preferably the method is for treating or preventing unresectable and/or metastatic malignant gastrointestinal stromal tumor (GIST) or advanced and/or metastatic renal cell carcinoma (MRCC). Preferably the patient is a mammal, preferably a human.

### BRIEF DESCRIPTION OF THE ACCOMPANYING FIGURES

FIG. 1 describes the X-ray powder diffraction (XRPD) of sunitinib form II.

FIG. 2 describes the differential scanning calorimetry (DSC) of sunitinib form II.

FIG. 3 describes the thermogravimetric analysis (TGA) of sunitinib form II.

FIG. 4 describes the X-ray powder diffraction (XRPD) of sunitinib form III.

FIG. 5 describes the differential scanning calorimetry (DSC) of sunitinib form III.

FIG. 6 describes the thermogravimetric analysis (TGA) of sunitinib form III.

### DETAILED DESCRIPTION OF THE INVENTION

As outlined above, the present invention provides two new crystalline forms of sunitinib designated as form II and form III, which are non-hygroscopic, polymorphically stable and have beneficial properties which avoid the problems associated with the prior art. The crystalline forms according to the invention can be used in the preparation of sunitinib malate or other sunitinib salts or polymorphs, or as APIs in a pharmaceutical product. In addition, convenient processes for the preparation of forms II and III have been provided and preferred embodiments of these processes are described below.

A preferred embodiment of the process for the preparation of crystalline forms II and III of sunitinib comprises the steps of:

(a) dissolving sunitinib in a solvent;
(b) causing form II or form III to precipitate from the solution obtained in step (a); and
(c) isolating the solid obtained in step (b).

When form II is desired, the solvent in step (a) is preferably a non-hydroxylic solvent. Preferably the non-hydroxylic solvent in step (a) is an ester. Most preferably, the ester is ethyl acetate.

When form III is desired, the solvent in step (a) is preferably a non-hydroxylic solvent. Preferably the solvent is a ketone. More preferably, the solvent is acetone. In alternative embodiments relating to the preparation of form III, the solvent is an alcohol, more preferably a C₁-C₆ alcohol. Most preferably, the solvent is isopropyl alcohol (IPA), but alternative alcohols may comprise methanol, propan-1-ol etc.

In preferred processes, complete dissolution of the sunitinib is indicated when a clear solution from step (a) is obtained. The clear solution is preferably obtained by dissolving the sunitinib in the relevant solvent at reflux temperature. It is within the skill set of the skilled person to determine the reflux temperatures of the preferred solvents.

The solution, however obtained, may preferably be filtered at this stage in order to further remove particulate impurities that may be present. The inventors have found that filtering under conditions of reduced pressure, preferably under conditions of a vacuum or partial vacuum, is particularly advantageous.

Causing the desired crystalline form to precipitate from the solution obtained in step (a) as required in step (b) can be achieved in any of a number of ways by the skilled person.

The inventors have found that cooling the solution will cause the desired crystalline form to precipitate from the solution. The skilled person will realize that the solution can be cooled to ambient temperature when the solution has been heated to effect dissolution of the sunitinib or indeed below that. The inventors have found that cooling to between about 0-20° C., preferably to between about 0-10° C., most preferably to between about 0-5° C., is particularly advantageous. The desired crystalline form may also be caused to precipitate for example by stirring the solution or by cooling the solution, even in those embodiments wherein dissolution of the sunitinib was not effected by heating, to below ambient temperature, preferably to between about 0-10° C., most preferably to between about 0-5° C.

A combination of stirring and allowing the solution to cool can also be employed. Stirring of the solution to effect precipitation may also be employed in those embodiments wherein the solution has been heated to effect dissolution. In these embodiments, it is envisaged that the stirring will be carried out during cooling or indeed once the solution has cooled. In any case, the stirring conditions may be varied and still remain within the scope of the invention.

The solid crystalline product obtained can then be isolated as required in step (c) by any means common in the field or known to the skilled artisan. Preferably the solid is washed with the same solvent as utilized in step (a). Thus, for example when the solvent used is ethyl acetate in the preparation of form II, it is preferred that the solid is washed with ethyl acetate. In one embodiment, the solid is obtained by evaporation of the solvent under ambient conditions. However, in a particularly preferred embodiment, the solid product is filtered and dried. Preferably the product is dried at a temperature that does not induce conversion of the crystalline form or causes the resultant crystalline form to degrade. The inventors have found that drying the product at between about 30-50° C., preferably at about 40° C., is advantageous. Preferably, in certain embodiments, the solid product is dried under vacuum or partial vacuum, most preferably at about 40° C. until a constant weight is obtained.

The processes of the invention provide forms II and III in a particularly pure form. In certain embodiments, there are provided crystalline forms II or III of sunitinib having a chemical purity of greater than 99%, preferably greater than 99.3%, more preferably greater then 99.4%, even more preferably greater than 99.5%, yet more preferably greater than 99.6%, yet more preferably still greater than 99.7%, most preferably greater than 99.9%, preferably as measured by HPLC.

As mentioned previously the sunitinib forms II or III according to the invention may be used as intermediates in the preparation of salts of sunitinib. Non-limiting examples include the hydrochloride, sulfate, carbonate, lactate, tartrate, malate, maleate or succinate salts. The salts may be prepared in any way known to the skilled person, but generally preparation of the salts involves contacting the sunitinib form II or III according to the invention with an appropriate acid. In particularly preferred embodiments, the acid is malic acid, but in alternative embodiments the salt prepared may be any pharmaceutically acceptable salt or indeed any salt useful in the preparation of a pharmaceutically acceptable form of sunitinib. The sunitinib form II or III may also be useful in the preparation of advantageous hydrates and solvates, by contacting the sunitinib form II or III according to the invention with water/aqueous solvent or a desired solvent respectively under appropriate conditions. The preparation of said hydrates, solvates and salts is well within the skill set of the skilled person to achieve and should be considered to be within the scope of the invention. It is also envisaged that the sunitinib forms II or III according to the invention may be used as intermediates in the preparation of other polymorphic forms. For example, WO 2003/016305 discloses methods for the preparation of the malate salt of sunitinib; the disclosure is incorporated herein by reference.

Accordingly, there is provided a process for preparing sunitinib malate, comprising reacting sunitinib form II or III according to the invention with malic acid. In a particularly preferred embodiment, the malic acid is L-malic acid or alternatively is D-malic acid.

A further aspect of the invention provides a composition comprising a pharmaceutically effective amount of one or more novel crystalline form(s) according to the invention or prepared according to the invention and further comprising one or more pharmaceutically acceptable excipient(s).

In a preferred embodiment, a pharmaceutical composition is provided comprising sunitinib malate prepared according to the invention. Further preferred embodiments provide a pharmaceutical composition comprising sunitinib malate prepared according to the invention for use in the treatment or prevention of cancer and/or tumors, preferably for the treatment or prevention of unresectable and/or metastatic malignant gastrointestinal stromal tumor (GIST) or advanced and/or metastatic renal cell carcinoma (MRCC).

The pharmaceutical composition according to the invention can be a solution or suspension, but is preferably a solid oral dosage form. Preferred oral dosage forms in accordance with the invention include tablets, capsules and the like which, optionally, may be coated if desired. Tablets can be prepared by conventional techniques, including direct compression, wet granulation and dry granulation. Capsules are generally formed from a gelatin material and can include a conventionally prepared granulate of excipients in accordance with the invention.

The pharmaceutical composition according to the present invention typically comprises one or more conventional pharmaceutically acceptable excipient(s) selected from the group comprising a filler, a binder, a disintegrant, a lubricant, and optionally further comprises at least one excipient selected from coloring agents, adsorbents, surfactants, film formers and plasticizers.

If the solid pharmaceutical formulation is in the form of coated tablets, the coating may be prepared from at least one film former such as hydroxypropyl methyl cellulose, hydroxypropyl cellulose or methacrylate polymers which optionally may contain at least one plasticizer such as polyethylene glycols, dibutyl sebacate, triethyl citrate, and other pharmaceutical auxiliary substances conventional for film coatings, such as pigments, fillers and others.

Preferably the pharmaceutical compositions according to one aspect of the invention are for use in treating or preventing disorders related to abnormal protein kinase (PK) activity. Such diseases include, but are not limited to, diabetes, hepatic cirrhosis, cardiovascular disease such as atherosclerosis, angiogenesis, immunological disease such as autoimmune disease, malignant gastrointestinal stromal tumor (MGIST) and metastatic renal cell carcinoma (MRCC).

The details of the invention, its objects and advantages are illustrated below in greater detail by non-limiting examples.

### EXAMPLES

### Example 1

### Preparation of Sunitinib Form II

Sunitinib (1 eq) was dissolved in ethyl acetate (25 vol) at reflux temperature to obtain a clear solution. The hot solution was filtered using a Buchner funnel under vacuum. The filtrate was cooled to ambient temperature between about 22-27° C., and a yellow to orange solid was obtained. The solid thus obtained was further filtered using a Buchner funnel under vacuum and washed with ethyl acetate. The solid was then dried under vacuum at about 40° C. for 3 hours to obtain crystal form II.

% Yield=76%

HPLC purity=99.13%

### Example 2

### Preparation of Sunitinib Form III

Sunitinib (1 eq) was dissolved in acetone (25 vol) at reflux temperature to obtain a clear solution. The hot solution was filtered through a Buchner funnel under vacuum. The filtrate was cooled to ambient temperature between about 22-27° C., and a yellow to orange solid was obtained. The solid thus obtained was further filtered using a Buchner funnel under vacuum and washed with acetone. The solid was then dried under vacuum at about 40° C. for 3 hours to obtain crystal form III.

% Yield=87%

HPLC purity=98.43%

### Example 3

### Alternative Preparation of Sunitinib Form III

Sunitinib (1 eq) was dissolved in IPA (25 vol) at reflux temperature to obtain a clear solution. The hot solution was filtered through a Buchner funnel under vacuum. The filtrate was cooled to ambient temperature between about 22-27° C., and a yellow to orange solid was obtained. The solid thus obtained was further filtered using a Buchner funnel under vacuum and washed with IPA. The solid was then dried under vacuum at about 40° C. for 3 hours to obtain crystal form III.

% Yield=85%

HPLC purity=98.77%

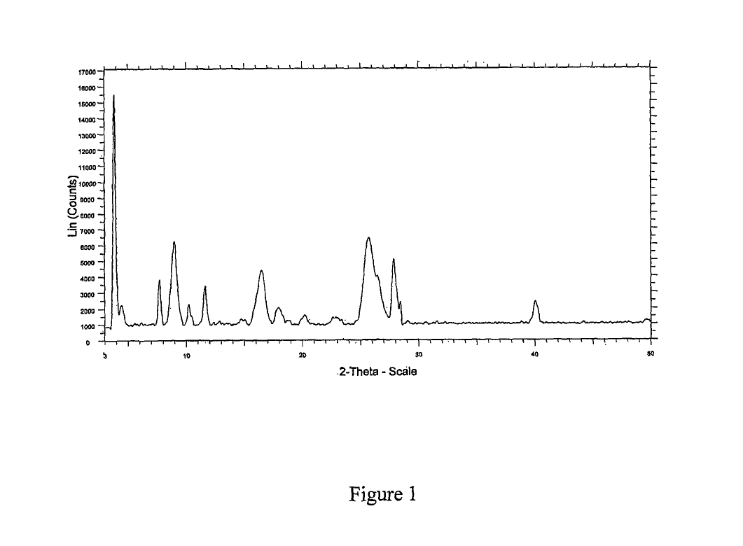

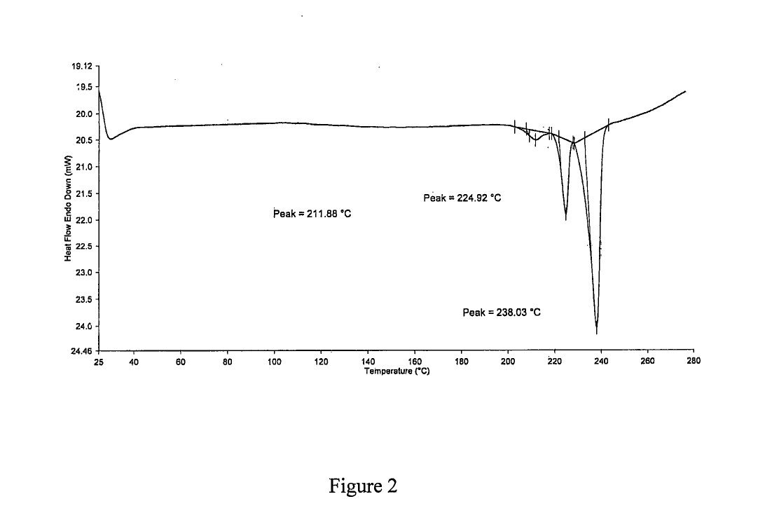

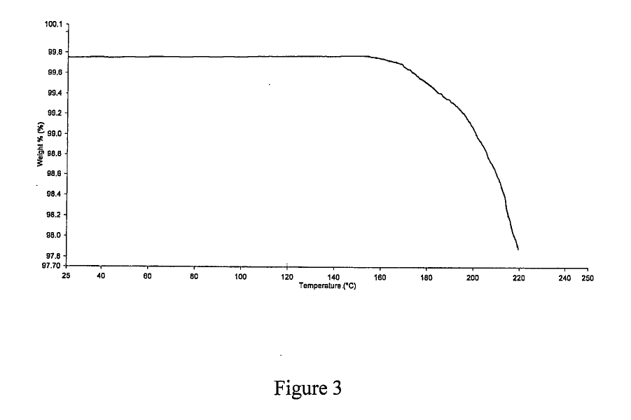

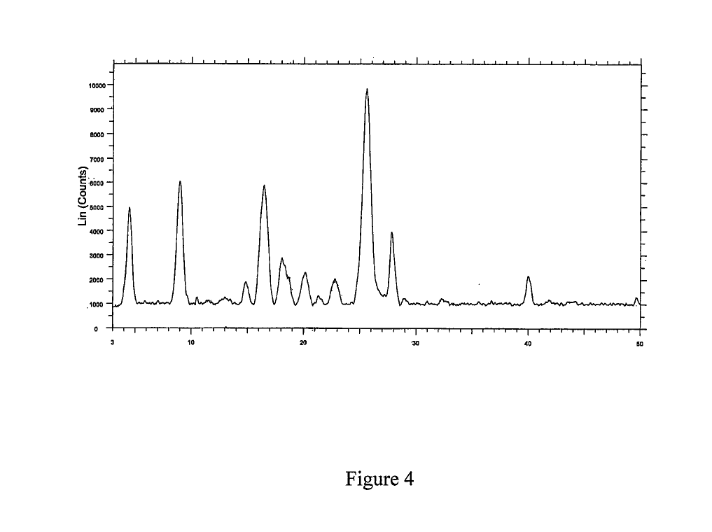

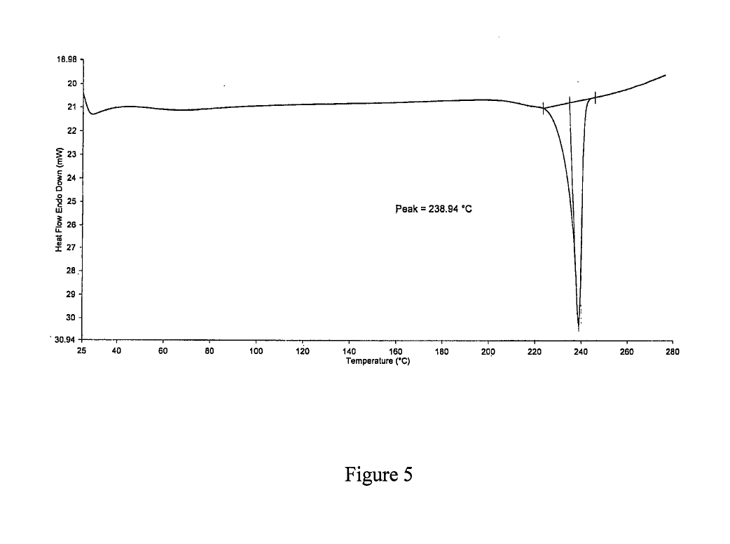

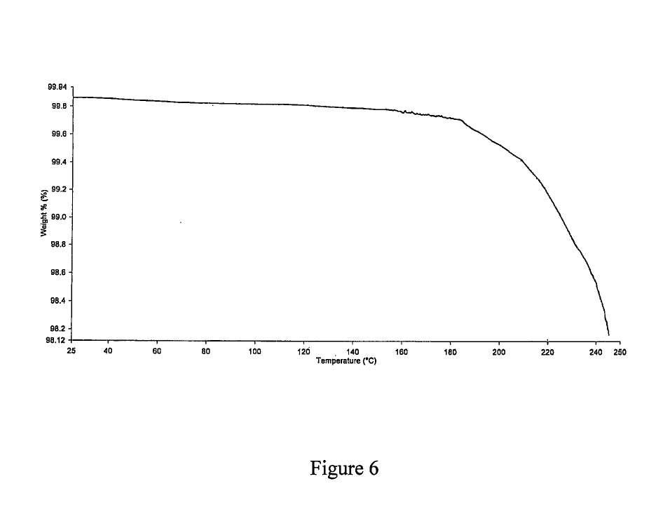

The crystalline forms prepared in the above examples were characterized by XRPD (shown in FIGS. 1 and 4), DSC (shown in FIGS. 2 and 5) and TGA (shown in FIGS. 3 and 6), and all shown to be the crystalline forms indicated.

The XRPDs were recorded on a Bruker D8 Advance Instrument, using Cu α-radiation as the X-ray source, with a 2θ range of from 3 to 50°, a step-size of 0.5° and a time/step of 1 sec.

The DSCs were recorded on a Perkin Elmer Pyris 6, with a temperature range of from 25° C. to 280° C. and a rate of heating of 10° C./min.

The TGAs were recorded on a Perkin Elmer Pyris 6, with a temperature range of from 25° C. to 250° C. and a rate of heating of 10° C./min.

It will be understood that the present invention has been described above by way of example only. The examples are not intended to limit the scope of the invention. Various modifications and embodiments can be made without departing from the scope and spirit of the invention, which is defined by the following claims only.

1.-49. (canceled)

50. Crystalline form II of sunitinib having a characteristic XRPD spectrum comprising two or more peaks with 2θ values at 3.76, 4.38, 7.65, 8.93, 10.17, 11.57, 16.46, 17.92, 20.15, 25.69, 26.39, 27.83, 28.40, 40.04 and 49.65±0.2 °2θ.

51. Crystalline form II of sunitinib according to claim 50, characterized by:
(i) a differential scanning calorimetry (DSC) with an endothermic peak at about 238° C.; and/or
(ii) a thermogravimetric analysis (TGA) loss of 0% over a range of between about 25-220° C.

52. Crystalline form II of sunitinib according to claim 50, having an HPLC purity of greater than 99%.

53. A process for the preparation of crystalline form II of sunitinib according to claim 50, comprising the steps of:
(a) dissolving sunitinib in a solvent;
(b) causing crystalline form II of sunitinib to precipitate from the solution obtained in step (a); and
(c) isolating the solid obtained in step (b).

54. A process according to claim 53, wherein:
(i) the solvent in step (a) is a non-hydroxylic solvent; and/or
(ii) the solvent in step (a) is an ester; and/or
(iii) the solvent in step (a) is ethyl acetate; and/or
(iv) the solvent in step (a) is heated to dissolve the sunitinib; and/or
(v) the solvent in step (a) is heated to reflux temperature to dissolve the sunitinib; and/or
(vi) the solution obtained in step (a) is filtered before carrying out step (b); and/or
(vii) the form II is caused to precipitate in step (b) by cooling the solution obtained in step (a); and/or
(viii) the form II is caused to precipitate in step (b) by cooling the solution obtained in step (a) to between about 0-5° C.; and/or
(ix) the solid obtained in step (b) is isolated by filtration; and/or
(x) the solid isolated in step (b) is washed with the solvent employed in step (a); and/or
(xi) the solid isolated in step (b) is dried until a constant weight is achieved.

55. Crystalline form III of sunitinib having a characteristic XRPD spectrum comprising two or more peaks with 2θ values at 4.40, 8.91, 10.45, 14.79, 16.40, 18.00, 18.59, 20.11, 22.76, 25.56, 27.81, 32.31, 40.02 and 49.71±0.2 °2θ.

56. Crystalline form III of sunitinib according to claim 55, characterized by:
(i) a differential scanning calorimetry (DSC) with an endothermic peak at about 239° C.; and/or
(ii) a thermogravimetric analysis (TGA) loss of 0% over a range of between about 25-220° C.

57. Crystalline form III of sunitinib according to claim 55, having an HPLC purity of greater than 99%.

58. A process for the preparation of crystalline form III of sunitinib according to claim 55, comprising the steps of:
(a) dissolving sunitinib in a solvent;
(b) causing crystalline form III of sunitinib to precipitate from the solution obtained in step (a); and
(c) isolating the solid obtained in step (b).

59. A process according to claim 58, wherein:
(i) the solvent in step (a) is a non-hydroxylic solvent (such as a ketone, such as acetone) or an alcohol (such as IPA); and/or
(ii) the solvent in step (a) is heated to dissolve the sunitinib; and/or
(iii) the solvent in step (a) is heated to reflux temperature to dissolve the sunitinib; and/or
(iv) the solution obtained in step (a) is filtered before carrying out step (b); and/or
(v) the form III is caused to precipitate in step (b) by cooling the solution obtained in step (a); and/or
(vi) the form III is caused to precipitate in step (b) by cooling the solution obtained in step (a) to between about 0-5° C.; and/or
(vii) the solid obtained in step (b) is isolated by filtration; and/or
(viii) the solid isolated in step (b) is washed with the solvent employed in step (a); and/or
(ix) the solid isolated in step (b) is dried until a constant weight is achieved.

60. A process for preparing sunitinib malate, comprising reacting crystalline form II of sunitinib according to claim 50 with malic acid.

61. A process according to claim 60, wherein the malic acid is L-malic acid or D-malic acid.

62. A process for preparing sunitinib malate, comprising reacting crystalline form III of sunitinib according to claim 55 with malic acid.

63. A process according to claim 62, wherein the malic acid is L-malic acid or D-malic acid.

64. A pharmaceutical composition comprising crystalline form II of sunitinib according to claim 50 and one or more pharmaceutically acceptable excipients.

65. A pharmaceutical composition comprising crystalline form III of sunitinib according to claim 55 and one or more pharmaceutically acceptable excipients.

66. A method of treating or preventing cancer or a tumor, the method comprising administering to a patient in need thereof a therapeutically or prophylactically effective amount of crystalline form II of sunitinib according to claim 50.

67. A method of treating or preventing cancer or a tumor, the method comprising administering to a patient in need thereof a therapeutically or prophylactically effective amount of crystalline form III of sunitinib according to claim 55.

68. A method of treating or preventing cancer or a tumor, the method comprising administering to a patient in need thereof a therapeutically or prophylactically effective amount of a pharmaceutical composition according to claim 64.

69. A method of treating or preventing cancer or a tumor, the method comprising administering to a patient in need thereof a therapeutically or prophylactically effective amount of a pharmaceutical composition according to claim 65.

70. A method of treating or preventing unresectable and/or metastatic malignant gastrointestinal stromal tumor (GIST) or advanced and/or metastatic renal cell carcinoma (MRCC), the method comprising administering to a patient in need thereof a therapeutically or prophylactically effective amount of crystalline form II of sunitinib according to claim 50.

71. A method of treating or preventing unresectable and/or metastatic malignant gastrointestinal stromal tumor (GIST) or advanced and/or metastatic renal cell carcinoma (MRCC), the method comprising administering to a patient in need thereof a therapeutically or prophylactically effective amount of crystalline form III of sunitinib according to claim 55.

72. A method of treating or preventing unresectable and/or metastatic malignant gastrointestinal stromal tumor (GIST) or advanced and/or metastatic renal cell carcinoma (MRCC), the method comprising administering to a patient in need thereof a therapeutically or prophylactically effective amount of a pharmaceutical composition according to claim 64.

73. A method of treating or preventing unresectable and/or metastatic malignant gastrointestinal stromal tumor (GIST) or advanced and/or metastatic renal cell carcinoma (MRCC), the method comprising administering to a patient in need thereof a therapeutically or prophylactically effective amount of a pharmaceutical composition according to claim 65.
